(19) United States
(12) Patent Application Publication
(10) Pub. No.: US 2020/0197650 A1
(43) Pub. Date: Jun. 25, 2020

(54) FLEXIBLE AIRWAY AND SEAL AND PATIENT INTERFACE DEVICE INCLUDING SAME

(71) Applicant: KONINKLIJKE PHILIPS N.V., Eindhoven (NL)

(72) Inventor: PETER CHI FAI HO, PITTSBURGH, PA (US)

(21) Appl. No.: 16/712,480

(22) Filed: Dec. 12, 2019

Related U.S. Application Data
(60) Provisional application No. 62/783,235, filed on Dec. 21, 2018.

(51) Int. Cl. A61M 16/08; A61M 16/06

(52) U.S. Cl. CPC A61M 16/0875; A61M 2205/0216; A61M 16/0616

(57) ABSTRACT

A patient interface device for use in delivering a flow of a breathing gas to the airway of a patient includes a frame formed of a substantially rigid material and a flexible airway formed of a pliable material. The flexible airway includes: a first end having an aperture defined therein which is structured to be coupled to a gas delivery conduit for receiving the flow of breathing gas; a second end disposed opposite the first end and coupled to the frame, the second end having a sealing flap extending radially inward from a periphery of the second end which is structured to sealingly engage the face of the patient about the airway of the patient; and a wall portion extending between the first end and the second end, the wall portion defining a passage adapted to convey the flow of breathing gas from the first end to the second end.

### CROSS-REFERENCE TO RELATED APPLICATIONS

This patent application claims the priority benefit under 35 U.S.C. § 119(e) of U.S. Provisional Application No. 62/783,235, filed on Dec. 21, 2018, the contents of which are herein incorporated by reference.

### BACKGROUND OF THE INVENTION

### 1. Field of the Invention

The present invention relates to non-invasive ventilation and pressure support systems wherein a patient interface device is used to deliver a flow of breathing gas to a patient and, more particularly, to a patient interface device including a flexible airway formed of a pliable material and a frame operably coupled to the flexible airway. The present invention also pertains a flexible airway for use in delivering a flow of breathing gas to a patient.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve a gas flow generator to produce a flow of breathing gas, and the placement of a patient interface device including a mask component on the face of a patient. The gas flow generator produces positive air pressure by taking air in from the surroundings and spinning a fan to push the air out of the machine, through a delivery conduit, and into the patient interface device to be delivered to the patient.

Traditional cushion members for patient interface devices include a sealing portion that is structured to engage the face of the patient in order to provide a seal therewith. Known sealing portions suffer from a number of drawbacks, such as applying too much pressure to certain areas of the face, resulting in red marks and a less than ideal fit. Furthermore, current injection molding techniques limit the ability to incorporate thin films in the sealing portions. Additionally, in known sealing portions that are made of fabric materials, gas flow leaks often occur as a result of the stitching between the fabric and the body of the cushion member. Moreover, employing a sealing portion entirely constructed of a fabric material is generally not practicable, as current fabric materials do not allow gas impermeability.

### SUMMARY OF THE INVENTION

As one aspect of the present invention a patient interface device for use in delivering a flow of a breathing gas to the airway of a patient is provided. The patient interface device comprises: a frame formed of a substantially rigid material; and a flexible airway formed of a pliable material, the flexible airway comprising: a first end having an aperture defined therein and being structured to be coupled to a gas delivery conduit for receiving the flow of breathing gas; a second end disposed opposite the first end and coupled to the frame, the second end having a sealing flap extending radially inward from a periphery of the second end which is structured to sealingly engage the face of the patient about the airway of the patient; and a wall portion extending between the first end and the second end, the wall portion defining a passage adapted to convey the flow of breathing gas from the first end to the second end.

The second end of the flexible airway may be coupled to the frame about the entire periphery of the second end of the flexible airway. The second end of the flexible airway may be coupled to the frame only at discrete locations along the periphery of the second end of the flexible airway. The frame may comprise an inner edge and an outer edge, the outer edge disposed rearward, and radially outward, from the inner edge, wherein the first end of the flexible airway is disposed at or about the inner edge of the frame; and wherein the second end of the flexible airway is coupled at or about the outer edge of the frame. The inner edge of the frame may define an aperture in which a portion of the flexible airway is disposed. The first end of the flexible airway may be coupled to the inner edge of the frame. The first end of the flexible airway may be coupled in the aperture by a folding back coupling arrangement. The wall portion may pass through the aperture and the first end may be coupled to the conduit. The wall portion may comprise at least one exhalation port structured to convey a gas exhaled from the patient. The frame may comprise a number of fastening mechanisms structured to couple with a headgear for securing the patient interface device to the head of the patient. The frame may comprise at least one opening defined therein which is structured to allow passage of gas therethrough from a cavity between the flexible airway and the frame. The pliable material may comprise one of: a thin polymer material or a woven fabric material. The frame may comprise a thin, ring-shaped member.

As another aspect of the invention, a flexible airway for use in a patient interface device for delivering a flow of a breathing gas to the airway of a patient is provided. The flexible airway comprises: a first end structured to be coupled to a gas delivery conduit for receiving the flow of breathing gas, the first end defining an aperture; a second end disposed opposite the first end, the second end having a sealing portion extending radially inward from the second end which is structured to sealingly engage the face of the patient about the airway of the patient; and a wall portion extending between the first end and the second end, the wall defining a passage adapted to convey the flow of breathing gas from the first end to the second end.

The wall portion may comprise at least one exhalation port structured to convey a flow of breathing gas exhaled from the patient. The flexible airway may be formed of a thin polymer material. The flexible airway may comprise a woven fabric material.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1A is a front view of a patient interface device disposed on the face of a patient and a portion of a conduit shown connected to a gas flow/pressure generating system (shown schematically) to form a system adapted to provide a regiment of respiratory therapy to a patient according to one exemplary embodiment of the invention;

FIG. 1B is a front isometric view of the patient interface device of FIG. 1A also shown disposed on the face of a patient;

FIG. 2 is a rear view of the patient interface device shown in FIGS. 1A and 1B;

FIG. 3 is a sectional view of the patient interface device of FIG. 1A taken along line A-A of FIG. 1A, shown disposed on the face of a patient;

FIG. 4 is an exploded side view of the patient interface device of FIG. 1A;

FIGS. 5A and 5B are side views of a patient interface device including a flexible airway coupled to a fluid coupling conduit according to one exemplary embodiment of the invention;

FIG. 6A is a lower front view of a frame according to one exemplary embodiment of the invention;

FIG. 6B is a side view of the frame depicted in FIG. 6A;

FIG. 7A is a front view of a patient interface device disposed on the face of a patient and a portion of a conduit shown connected to a gas flow/pressure generating system (shown schematically) to form a system adapted to provide a regiment of respiratory therapy to a patient according to one exemplary embodiment of the invention;

FIG. 7B is a side view of the patient interface device shown in FIG. 7A; and

FIG. 7C is a cross sectional view of the patient interface device of FIGS. 7A and 7B taken along line B-B of FIG. 7A.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are coupled directly in contact with each other (i.e., touching). As used herein, “fixedly coupled” or “fixed” means that two components are coupled so as to move as one while maintaining a constant orientation relative to each other.

As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, left, right, upper, lower, front, back, on top of, and derivatives thereof, relate to the orientation of the elements shown in the drawings and are not limiting upon the claims unless expressly recited therein.

As used herein, the term “fabric” shall mean a material consisting of a network of interlaced or otherwise entangled natural or artificial fibers made by, for example and without limitation, weaving, knitting, spreading, crocheting, or bonding (e.g., by chemical, mechanical, heat or solvent treatment) the fibers to form the network, and may include, for example, and without limitation, woven and nonwoven fabric materials.

As used herein, the phrase “mechanical bond” shall mean a bond formed as a result of the curing (i.e., solidifying) of a material selected from the group consisting of a monomer, a polymer, and a mixture of a monomer and a polymer (e.g., without limitation, silicone) to a fabric material. For example and without limitation, a bond formed when a viscous silicone material flows into fibers of a fabric material and is thereafter cured is a mechanical bond. A connection formed when a fabric material is stitched to a silicone material is not a mechanical bond.

As used herein, the phrase “chemical bond” shall mean a bond formed as a result of the curing (i.e., solidifying) of a first material to a second material, where each of the first and second materials is made of a monomer, a polymer, or a mixture of a monomer and a polymer.

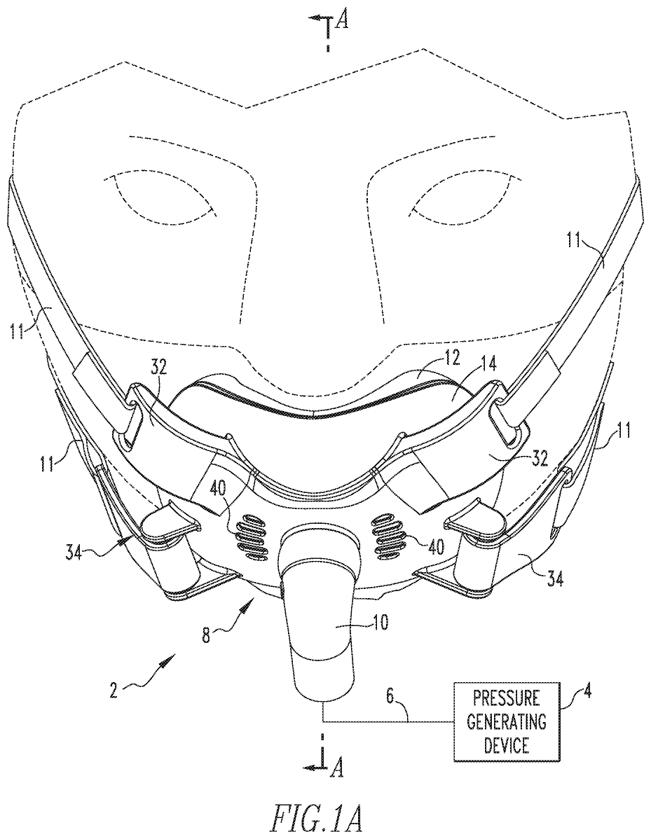

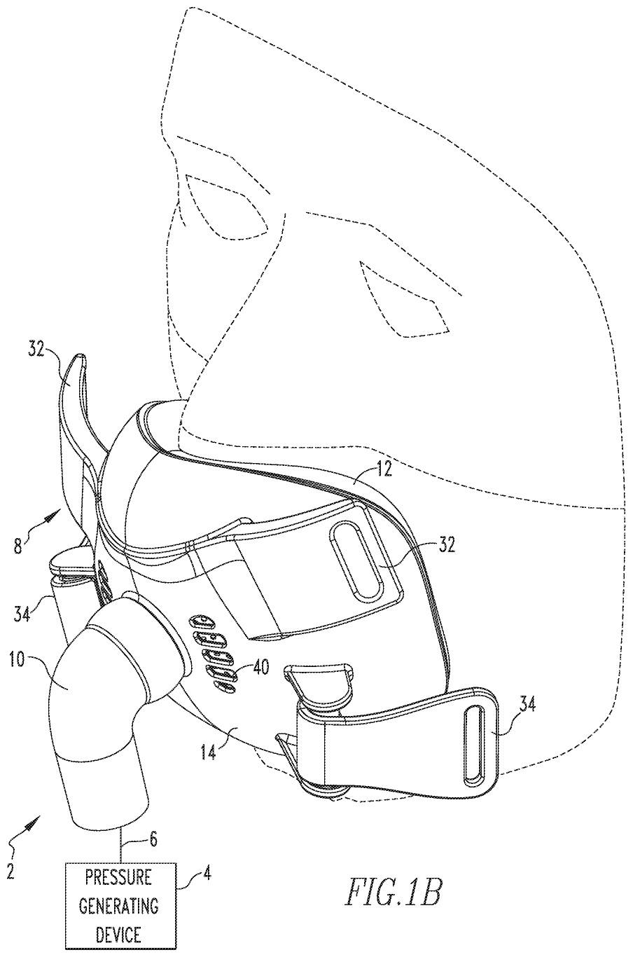

A system 2 adapted to provide a regimen of respiratory therapy to a patient according to one exemplary embodiment of the invention is generally shown in FIGS. 1A and 1B. System 2 includes a pressure generating device 4 (shown schematically), a delivery conduit 6 (shown schematically), a patient interface device 8 having a fluid coupling conduit 10, and a headgear 11 (only portions of straps thereof are shown). Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10. Delivery conduit 6 and patient interface device 8 are often collectively referred to as a patient circuit. In the exemplary embodiment illustrated in FIGS. 1A and 1B, fluid coupling conduit 10 is an elbow connector, however, it is to be appreciated that other suitable couplings may be employed without varying from the scope of the present invention. It is also to be appreciated that headgear 11 is provided solely for exemplary purposes and that any suitable headgear arrangement may be employed without varying from the scope of the present invention.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a patient or for elevating a pressure of gas at an airway of the patient, including the pressure support systems summarized above and non-invasive ventilation systems.

In the exemplary embodiment illustrated in FIGS. 1A and 1B, patient interface device 8 is depicted as a nasal/oral mask which includes a generally pliable flexible airway 12 coupled to a generally rigid frame 14, both of which are coupled to conduit 6 via fluid coupling conduit 10. However, it is to be appreciated that other types of patient interface devices, such as, without limitation, full face mask, nasal mask, nasal pillow cushion and conventional oral-nasal mask which covers higher up to the nose bridge, which facilitates the delivery of the flow of breathing gas to the airway of the user, may be substituted for patient interface device 8 while remaining within the scope of the present invention. It is also to be appreciated that conduit 6 may be directly coupled to patient interface device 8 without the use of any intermediary coupling, such as conduit 10.

Flexible airway 12 may be formed of any pliable material (e.g., without limitation, silicone, one or more polymer films, plastics, one or more fabrics, etc.). Frame 14 may be formed of a substantially rigid material (e.g., without limitation, one or more plastics) so as to provide structural support and a predetermined amount of rigidity to flexible airway 12.

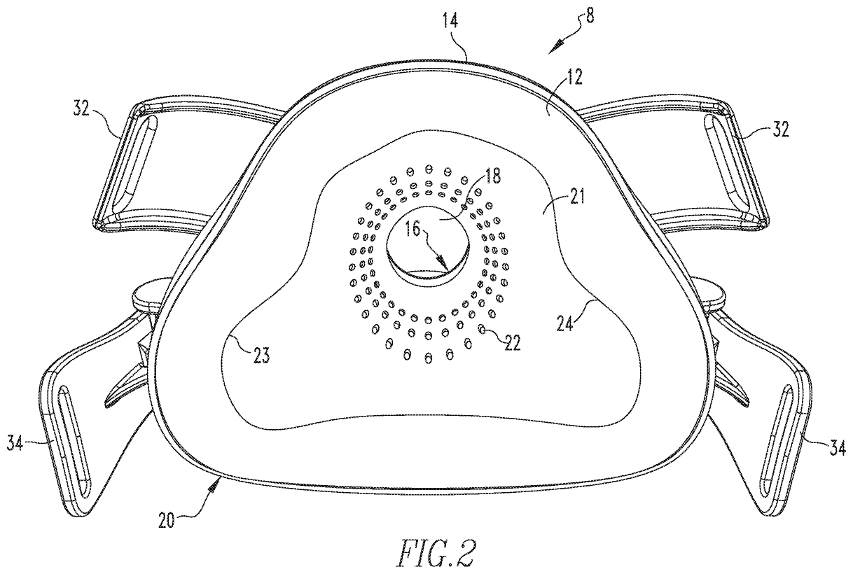

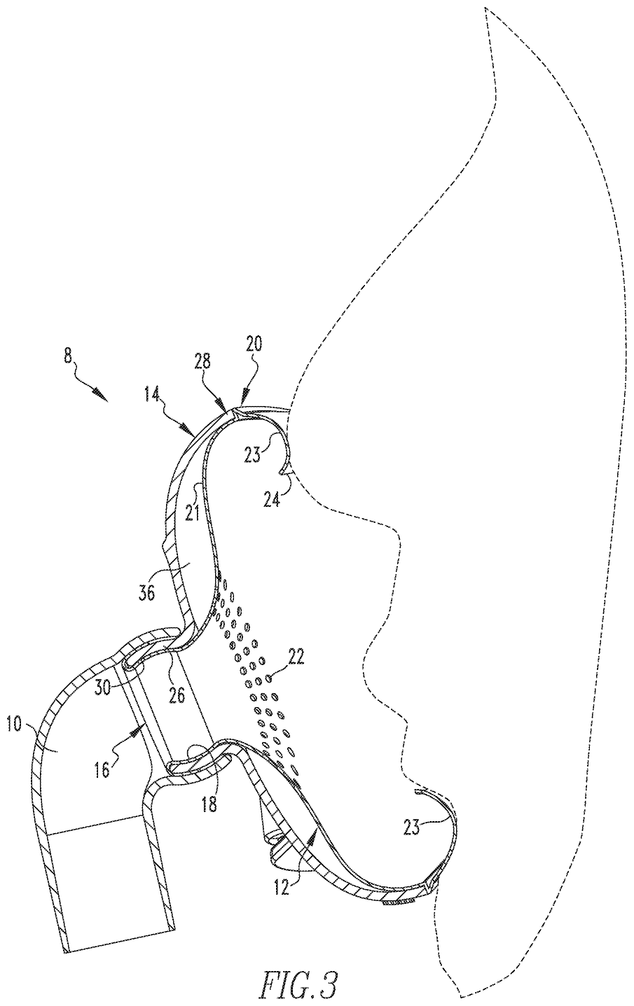

Referring to FIGS. 2-4, flexible airway 12 may be of a unitary structure and extends generally radially outward and rearward from a first end 16 (described in greater detail below in conjunction with FIG. 3), having an aperture 18 defined therein, to an opposite, second end 20 (described in greater detail below in conjunction with FIG. 3). Flexible airway 12 further includes a wall portion 21 which extends between first end 16 and second end 20 which defines a passage therebetween which is adapted to convey a flow of breathing gas received at first end 16 to second end 20. A number of exhaust ports 22 may be provided in wall portion 21 to facilitate exhaust of gases exhaled by the patient. Alternatively, wall portion 21 may be formed from an air permeable fabric which allows for exhaust gases to pass therethrough, or an exhaust arrangement may be provided on one or more of conduits 6, 10.

Flexible airway 12 further includes a sealing portion 23 provided along a periphery of second end 20. In the example embodiment illustrated in the figures sealing portion 23 is a sealing flap which generally extends radially inward from the periphery of second end 20 which is structured to sealingly engage the face of a patient about an airway or airways of the patient. Sealing flap 23 runs continuously along second end 20 such that it forms a perimeter defining a patient opening 24 at second end 20. In the illustrated example embodiment aperture 18 is substantially smaller than patient opening 24 defined by sealing flap 23, it is to be appreciated that one or more of the individual and/or relative sizing of such elements may be varied for different applications without varying from the scope of the present invention. For example, patient opening 24 defined by sealing flap 23 may be substantially wider to accommodate a full face mask, or may be substantially narrower to accommodate a nasal mask. It is to be appreciated that although shown as a sealing flap in the illustrated example, sealing portion 23 may be of other suitable patient sealing arrangement (e.g., without limitation, a cushion, nasal pillows, etc.) without varying from the scope of the present invention.

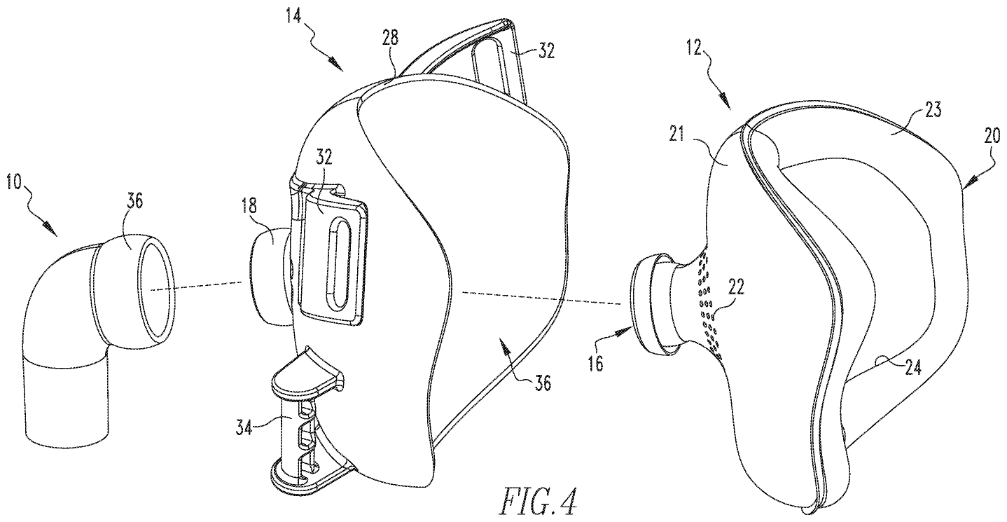

Continuing to refer to FIGS. 3 and 4, frame 14 is a generally concave shaped structure which extends from an inner edge 26 to an outer edge 28 which is disposed rearward and radially outward from inner edge 26. In the illustrated example embodiment, inner edge 26 defines an aperture 30 in which a portion of flexible airway 12 is disposed. Outer edge 28 preferably is shaped so as to generally follow the contours of the patient's face adjacent to frame 14. In an example embodiment, the shape of outer edge 28 was formed using dimensional data obtained from the particular patient. Frame 14 includes a number of attachment mechanisms 32, 34 for use in coupling mask 14, and thus patient interface device 10, to headgear 11 such as previously discussed in regard to FIG. 1A. It is to be appreciated that attachment mechanisms 32 and 34 are provided for exemplary purposes only and that any suitable attachment mechanisms may be employed without varying from the scope of the present invention.

Referring to FIG. 3, second end 20 of flexible airway 12 is coupled to frame 14 generally at or about outer edge 28 of frame 14 such that frame 14 provides support to sealing portion 23 (i.e., sealing flap 23) about the entirety thereof. Such coupling between frame 14 and flexible airway 12 may be accomplished via mechanical or chemical means without varying from the scope of the present invention. Furthermore, such coupling may occur about the entire periphery of second end 20 of flexible airway 12 or only at discrete locations along the periphery of second end 20.

Flexible airway 12 may further be coupled to frame 14 at or about first end 16 of flexible airway 12. In the example illustrated in FIG. 3, first end 16 of flexible airway 12 is positioned in aperture 30 and coupled to inner edge 26 of frame 14 by a folding arrangement. Such folding arrangement coupling is accomplished by passing first end 16 of flexible airway 12 through aperture 30 and then folding the portion of flexible airway 12 which extends through aperture 30 back onto the portion of frame 14 which defines aperture 30. It is to be appreciated that such folding arrangement coupling is provided for exemplary purposes only and that other coupling arrangements may be utilized without varying from the scope of the present invention. In other embodiments of the present invention, flexible airway 12 may be not coupled to frame 14 at inner edge 26 and first end 16. Rather, first end 16 of cushion may simply extend beyond inner edge 26 of frame 14 and directly couple to conduit 6 or 10 without coupling at all to frame 14.

In the example embodiment of FIGS. 1-4, flexible airway 12 is coupled to frame 14 only at or about inner edge 26 and outer edge 28. As such, a cavity 36 (see FIG. 3) is defined between frame 14 and wall portion 21 of flexible airway 12. In order to provide for evacuation of any gases from cavity 36, e.g., exhalation gases from exhaust ports 22 of flexible airway 12, frame 14 may further include a number of frame vents 40 defined in frame 12 between inner edge 26 and outer edge 28.

From the foregoing discussion, it is to be appreciated that the coupling of frame 14 to flexible airway 12 at outer edge 28 allows for flexible airway 12 to have structural integrity without the need for a rubbery or silicone backing, as is common in the art. The coupling at outer edge 28 also allows for a variety of advantages. For instance, the coupling at outer edge 28 allows for flexible airway 12 to be significantly less thick than is common in the art for similar structures (e.g., as little as a single layer of a lightweight material such as supper thin silicone <0.25 mm thick, saran wrap type plastic sheet as thin as 12.5 μm (0.0125 mm)), while allowing flexible airway 12 to be securely attached to frame 14 in a manner which provides for a substantially sealed engagement with the patient.

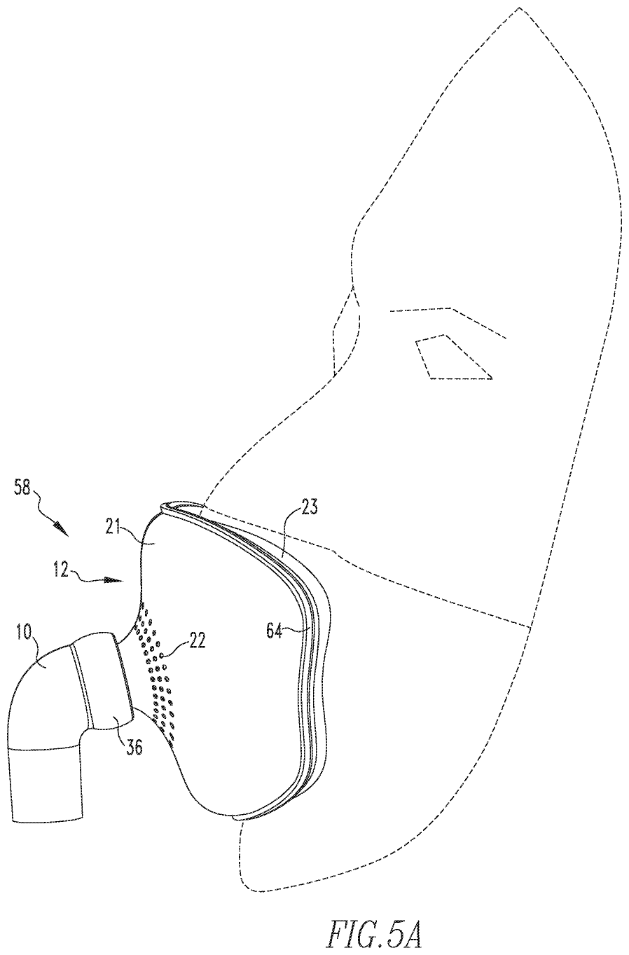

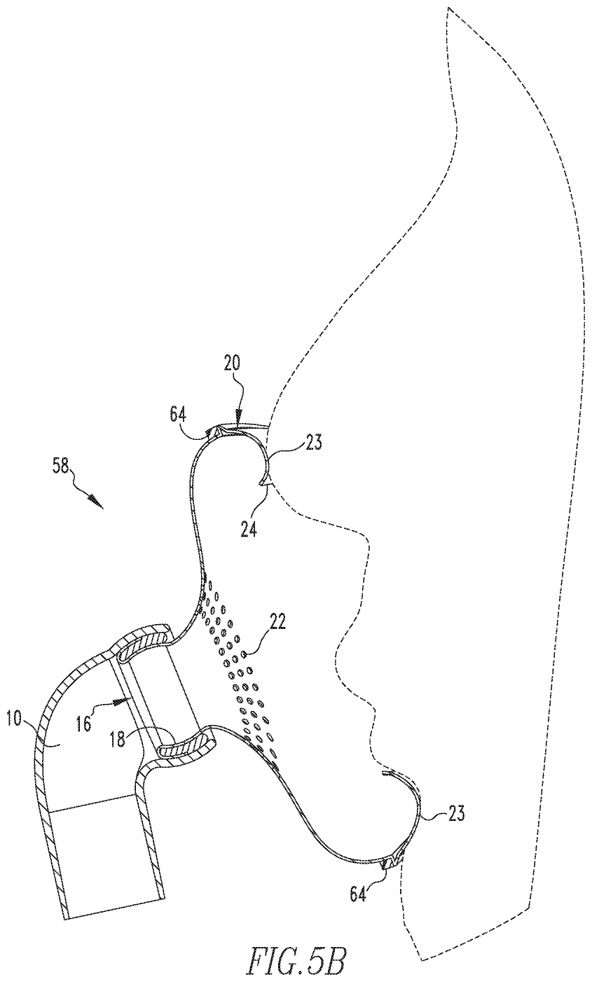

Referring now to FIGS. 5A and 5B, another patient interface device 58 in accordance with one example embodiment of the present invention which utilizes flexible airway 12 is illustrated. Patient interface device 58 utilizes a frame 64 which is generally merely a thin ring-shaped member shaped to generally mimic the contours of the patient's face adjacent to frame 64. Second end 20 of flexible airway 12 is coupled to frame 64 such that frame 64 provides support to sealing portion 23 (i.e., sealing flap 23) about the entirety thereof. Such coupling between frame 64 and flexible airway 12 may be accomplished via mechanical or chemical means without varying from the scope of the present invention. Furthermore, such coupling may occur about the entire periphery of second end 20 of flexible airway 12 or only at discrete locations along the periphery of second end 20. In such arrangement, frame 64 is merely intended to assist in sealing second end 20 of flexible airway 12 to the patient's face and not to support a delivery conduit or headgear attachments. Patient interface device 58 is generally intended only for short duration usage where either the patient or a caregiver would generally hold device 58 on the face of the patient.

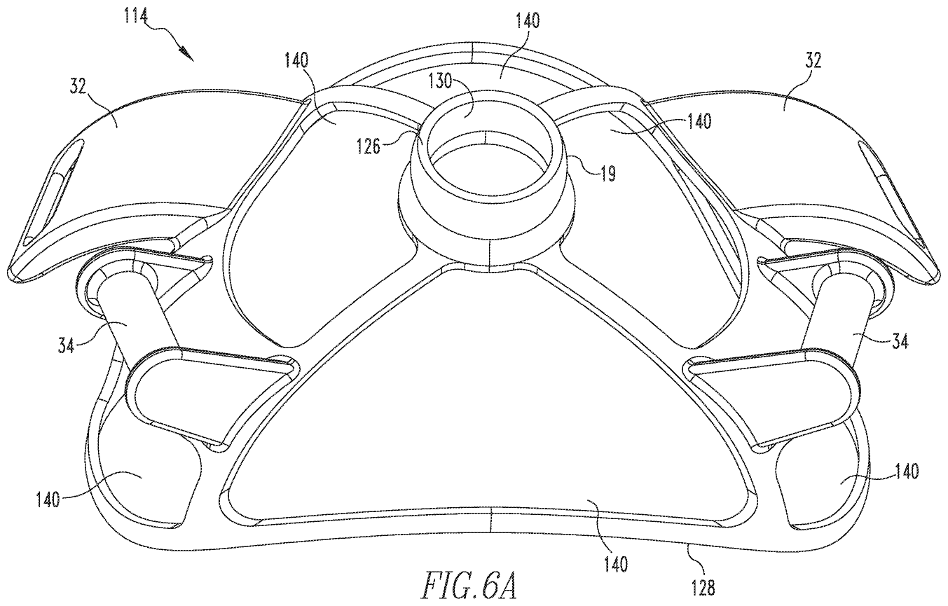

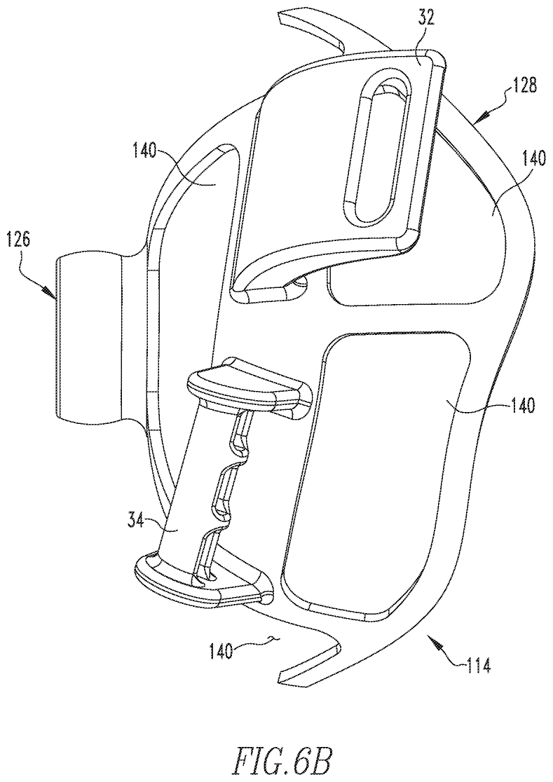

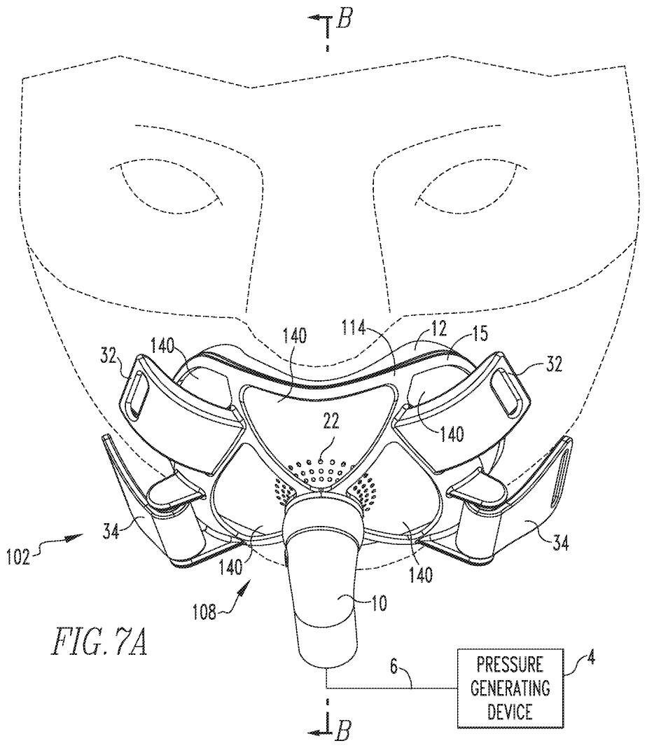

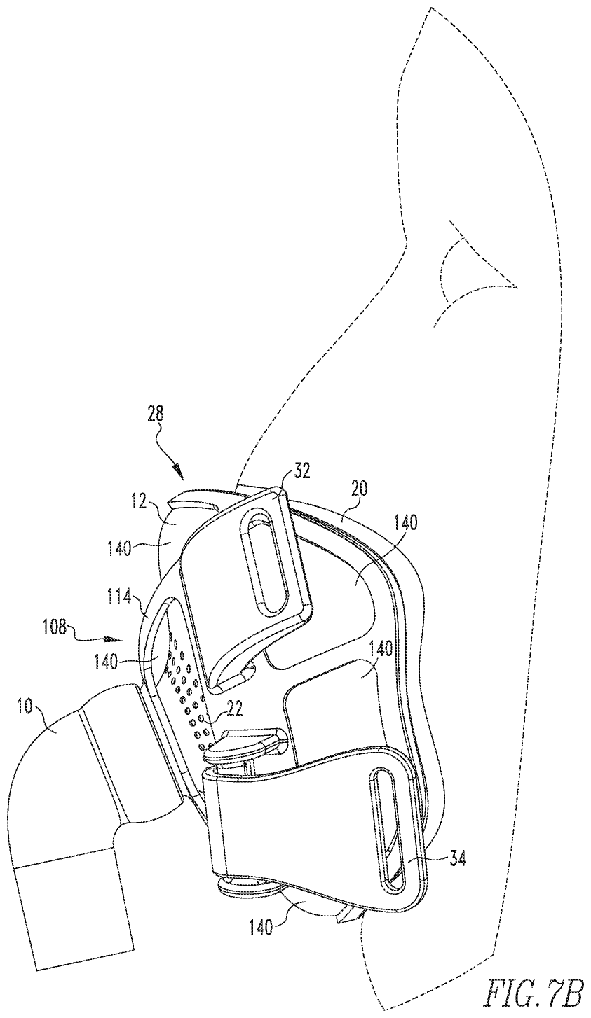

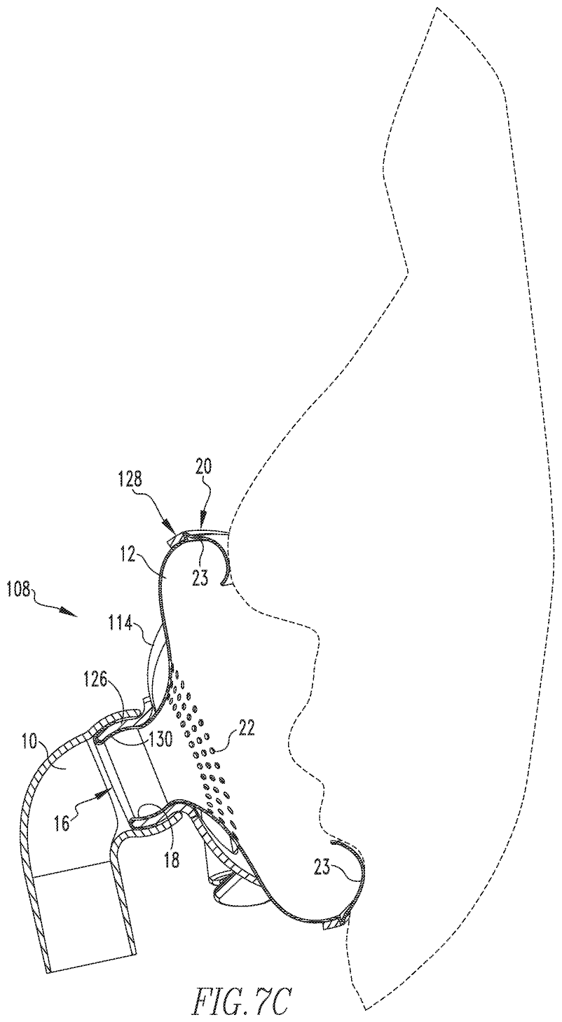

FIGS. 6A and 6B illustrate another example frame 114 in accordance with another example embodiment of the present invention which may be employed with a flexible airway in accordance with the present invention, such as flexible airway 12 previously described. Frame 114 has a substantially skeletal structure. More particularly, as depicted in FIGS. 6A and 6B, frame 114 includes a plurality of large openings 140 defined therein such that the amount of material between inner edge 126 and outer edge 128 of frame 114 is minimal, being large enough to still allow for attachment mechanisms, e.g., 32, 34, for coupling a suitable headgear. FIGS. 7A-7C show various views of such an alternative system 102 for providing a regimen of respiratory therapy to a patient according to one exemplary embodiment of the present invention that includes frame 114 in place of frame 14. Referring to FIG. 7A, openings 140 are substantially larger than exhalation ports 22, through which gas exhaled by a user may interface device 108 after passing through exhalation ports 22 of flexible airway 12. As shown in FIGS. 7B and 7C, flexible airway 12 does not engage or otherwise couple to frame 114 except at inner edge 126 and outer edge 128.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A patient interface device for use in delivering a flow of a breathing gas to the airway of a patient, the patient interface device comprising:
a frame formed of a substantially rigid material; and
a flexible airway formed of a pliable material, the flexible airway comprising:
a first end having an aperture defined therein and being structured to be coupled to a gas delivery conduit for receiving the flow of breathing gas;
a second end disposed opposite the first end and coupled to the frame, the second end having a sealing flap extending radially inward from a periphery of the second end which is structured to sealingly engage the face of the patient about the airway of the patient; and
a wall portion extending between the first end and the second end, the wall portion defining a passage adapted to convey the flow of breathing gas from the first end to the second end.

2. The patient interface device of claim 1, wherein the second end of the flexible airway is coupled to the frame about the entire periphery of the second end of the flexible airway.

3. The patient interface device of claim 1, wherein the second end of the flexible airway is coupled to the frame only at discrete locations along the periphery of the second end of the flexible airway.

4. The patient interface device of claim 1, wherein the frame comprises an inner edge and an outer edge, the outer edge disposed rearward, and radially outward, from the inner edge; wherein the first end of the flexible airway is disposed at or about the inner edge of the frame; and wherein the second end of the flexible airway is coupled at or about the outer edge of the frame.

5. The patient interface device of claim 4, wherein the inner edge of the frame defines an aperture in which a portion of the flexible airway is disposed.

6. The patient interface device of claim 4, wherein the first end of the flexible airway is coupled to the inner edge of the frame.

7. The patient interface device of claim 5, wherein the first end of the flexible airway is coupled in the aperture by a folding back coupling arrangement.

8. The patient interface device of claim 5, wherein the wall portion passes through the aperture, and wherein the first end is coupled to the conduit.

9. The patient interface device of claim 1, wherein the wall portion comprises at least one exhalation port structured to convey a gas exhaled from the patient.

10. The patient interface device of claim 1, wherein the frame comprises a number of fastening mechanisms structured to couple with a headgear 11 for securing the patient interface device to the head of the patient.

11. The patient interface of claim 1, wherein the frame comprises at least one opening defined therein which is structured to allow passage of gas therethrough from a cavity between the flexible airway and the frame.

12. The patient interface of claim 1, wherein the pliable material comprises one of: a thin polymer material or a woven fabric material.

13. The patient interface of claim 1, wherein the frame comprises a thin, ring-shaped member.

14. A flexible airway for use in a patient interface device for delivering a flow of a breathing gas to the airway of a patient, the flexible airway comprising:
a first end structured to be coupled to a gas delivery conduit for receiving the flow of breathing gas, the first end defining an aperture;
a second end disposed opposite the first end, the second end having a sealing portion extending radially inward from the second end which is structured to sealingly engage the face of the patient about the airway of the patient; and
a wall portion extending between the first end and the second end, the wall defining a passage adapted to convey the flow of breathing gas from the first end to the second end.

15. The flexible airway of claim 14, wherein the wall portion comprises at least one exhalation port structured to convey a flow of breathing gas exhaled from the patient.

16. The flexible airway of claim 14, wherein the flexible airway is formed of a thin polymer material.

17. The flexible airway of claim 14, wherein the flexible airway comprises a woven fabric material.
